(19) United States
(12) Patent Application Publication
(10) Pub. No.: US 2021/0244858 A1
(43) Pub. Date: Aug. 12, 2021

(54) SOFT TISSUE FILLER COMPOSITION WITH HYALURONIC ACID AND BENZYL ALCOHOL

(71) Applicant: Prollenium Medical Technologies, Inc., Aurora (CA)

(72) Inventors: Timothy Lee, Aurora (CA); Shadi Moghadam, Aurora (CA)

(21) Appl. No.: 17/227,714

(22) Filed: Apr. 12, 2021

Related U.S. Application Data
(63) Continuation-in-part of application No. 16/592,151, filed on Oct. 3, 2019.
(60) Provisional application No. 62/740,507, filed on Oct. 3, 2018.

(51) Int. Cl. A61L 27/54; A61L 27/20; A61K 31/045

(52) U.S. Cl. CPC A61L 27/54; A61L 27/20; A61L 2300/402; A61L 2300/216; A61L 2430/34; A61K 31/045

(57) ABSTRACT

A soft tissue filler composition including a cross-linked hyaluronic acid and a benzyl alcohol. The benzyl alcohol can be present at a concentration between about 0.1% and about 2.0% by weight of the composition.

### CROSS REFERENCE TO RELATED APPLICATIONS

The present application is a continuation in part of U.S. Ser. No. 16/797,130 filed Feb. 21, 2020. The present application also claims the priority of USSN 63/068,444 filed Aug. 21, 2020.

### FIELD OF THE INVENTION

The present invention relates to soft tissue filler compositions.

### BACKGROUND OF THE INVENTION

It is well known to use a combination of hyaluronic gel and lidocaine as a soft tissue filler composition.

### SUMMARY OF THE INVENTION

The present application discloses a soft tissue filler composition that includes, by weight:

| | |
|---|---|
| 96.5% | water; |
| 2.5% | volumizing agent; |
| 0.92% | osmotic pressure agent; |
| 0.017% | buffering agent; |
| trace | cross-linker; and |
| 1% | benzyl alcohol. |

According to another aspect of the invention the volumizing agent can be a Poly (β-gluconic acid-[1→3]-β-N-acetylglucosamine-[1→4].

According to another aspect of the invention the osmotic pressure agent can be 0.9% sodium chloride and 0.02 potassium chloride.

According to another aspect of the invention the buffering agent can be 0.014% sodium dihydrogen phosphate monohydrate salt and 0.003% potassium dihydrogen phosphate.

According to another aspect of the invention the cross-linker can be a butanediol diglycidyl ether (BDDE).

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is a graphical depiction of a dermal filler infused with effective amounts of benzyl alcohol and lidocaine and stored at 55° C. for accelerated aging over a period of weeks and depicting an elasticy or G prime (G′) dropping most significantly for the lidocaine infused product, and least for the benzyl alcohol infused product.

### DETAILED DESCRIPTION OF THE PREFERRED EMBODIMENTS

With reference to the attached illustrations, the present invention discloses a soft tissue filler composition with hyaluronic acid and benzyl alcohol. According to one non-limiting preferred embodiment, the composition includes, by weight:

| | |
|---|---|
| 96.5% | water; |
| 2.5% | Poly (β-gluconic acid-[1 →3]-β-N-acetylglucosamine-[1→4]; |
| 0.9% | sodium chloride; |
| 0.02 | potassium chloride; |
| 0.014% | sodium dihydrogen phosphate monohydrate salt; |
| .003% | potassium dihydrogen phosphate; |
| trace | BDDE; and |
| 1% | benzyl alcohol. |

The anesthetic effect of the benzyl alcohol has a shorter duration than lidocaine. In terms of pain assessment, it has been found that benzyl alcohol was significantly less painful than injecting plain lidocaine and in combination with lidocaine (1%) reduces injection pain as well as anesthesia duration. (Williams, J. M. and Howe, N. R. 1994. Benzyl Alcohol Attenuates the Pain of Lidocaine Injections and Prolongs Anesthesia. J Dermatol Surg Oncol, 20: 730-733).

Surprisingly, it has also been found that benzyl alcohol is better than lidocaine in terms of stability.

In this regard reference is made to Table 1 which shows dermal filler of the type sold by Prollenium Medical Technologies Inc. under the brand REVANESSE® infused with amounts of benzyl alcohol and lidocaine as shown. The benzyl alcohol product was held for 12 weeks at 55° C. which approximates 2 years shelf life; the amount of benzyl alcohol degraded by only 5.75%. In contrast, the amount of lidocaine degraded by 11.61% over 12 weeks at 45° C., which approximates to 1 year shelf life.

TABLE 1

| | Benzyl | |
|---|---|---|
| | alcohol | Lidocaine |
| Time | mg/g | mg/g |
| (weeks) | (55° C.) | (45° C.) |
| 0 | 8.7 | 3.1 |
| 4 | 8.7 | 2.9 |
| 8 | 8.6 | 2.82 |
| 12 | 8.2 | 2.74 |

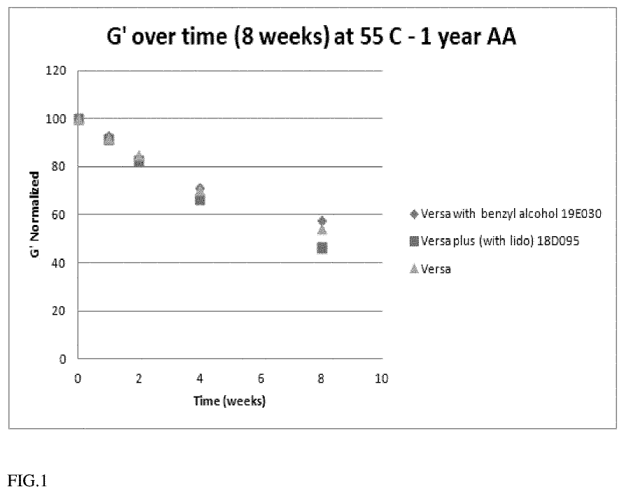

Similar improvements in G′ (as generally identified describing how the filler is able to retain its shape when a force is applied) were observed, as shown in FIG. 1. By general rule, fillers with lower G′ are softer, and spread through tissues easier.

This Figure shows dermal filler of the type sold by Prollenium Medical Technologies Inc. under the brand VERSA infused with effective amounts of benzyl alcohol and lidocaine and stored at 55° C. for accelerated aging. Over 8 weeks, G′ dropped most significantly for the lidocaine infused product, and least for the benzyl alcohol infused product.

Further testing was done of a hyaluronic dermal filler with a viscosity of about 3000 Pa.s, an HA particle size of 250-300 microns and 25 mg/ml, as shown below in Table 2 below.

| Time | | Benzyl | Benz- | Soluble | | | Extrusion | | | BDDE | | | | Tem- |
|---|---|---|---|---|---|---|---|---|---|---|---|---|---|---|
| Point | [HA] | Alcohol | aldehyde | HA | | [symbol] smo | Force | Vis- | G′ | Conc. | | E [symbol] | Visual | perature |
| (weeks) | mg/g | (mg/g) | (%) | (%) | pH | (mOs [symbol] ) | (lbs) | cosity | (mPa) | (ppm) | Sterility | (EU/mL) | inspection | profile |
| 0 | 25.7 | 9.5 | 0.03 | N/A | 7.3 | 302 | 2.6 | 3427 | 1.82E+05 | 0.3 | sterile | <0.075 | pass | N/A |
| 26 | 25.8 | 9. [symbol] | 0.01 | 43.6 | 7.3 | 308 | 2.8 | 3123 | 1.86E+05 | 0.1 | Sterile | <0.075 | arranged | N/A |
| 39 | 26. [symbol] | [symbol] | N/A | N/A | 7.3 | 286 | 3.4 | 3349 | 1.79E+05 | N/A | N/A | N/A | pass | yes |
| 52 | 26.9 | 9.0 | 0.01 | 44. [symbol] | 7.3 | 313 | 3.5 | 3175 | 1.75E+05 | 0.1 | sterile | <0.075 | 97.1 ** | yes |
| Spec | 22-28 | 8.1-9.9 | ≤1.0% of | Report | 6.8- | 260-360 | ≤5.0 | 1250- | Report | <2 | Sterile | <0.5 | Pass | N/A |
| | | | benzyl | | 7.6 | | | 3750 | | | | | | |
| | | | alcohol | | | | | | | | | | | |

[symbol]

indicates data missing or illegible when filed

Without intending to be bound by theory, it is contemplated that this combination of features will permit the practitioner to make facial assessments on the HA product and administer further injections within a single visit of typical duration. To explain: because the benzyl alcohol remains relatively stable when admixed, relatively small amounts of benzyl can be used in the formulation. This keeps costs down, and also ensures that the practitioner is not injecting excess anaesthetic into the patient, the latter giving the practitioner confidence to administer additional doses. The reduced pain provided by the benzyl alcohol gives the patient confidence to request additional doses. In totality, the use of benzyl alcohol as an anaesthetic should permit the practitioner to be more effective in practise, giving patients a better outcome.

Whereas a specific composition is mentioned, variations in the components other than benzyl alcohol are possible and will be obvious to persons of ordinary skill in the art.

For example, volumizing agents other than Poly (β-gluconic acid-[1→3]-β-N-acetylglucosamine-[1→4] can be used, and in differing amounts.

As well, osmotic pressure agents other than sodium chloride and potassium chloride can be used, and different amounts can also be used.

Additionally, buffering agents other than sodium dihydrogen phosphate monohydrate salt and potassium dihydrogen phosphate can be used, and different amounts can also be used.

Additionally, since benzyl alcohol has been shown in combination with other anesthetics to mitigate pain experience upon injection, it can be used with at least any of one local anesthetic or from a local anesthetic from the group of benzocaine, chloroprocaine, procaine, etidocaine, aptocaine, chlorobutanol, diamocaine, dyclonine, guafecainol, polidocanol, peivacaine, prilocaine, articaine, bupivacaine, ropivacine, tetracaine and salts therof and isolated isomer thereof.

Yet further, cross-linkers other than BDDE can be used, and in different amounts.

Moreover, the amount of benzyl alcohol can be varied: a useful range of 0.1 to 2.0% by weight of composition is contemplated. Based on the safety assessment of the anesthetic, it has been shown in literature that a 0.05% to a 5% solution was considered somewhat effect. It was noted that a 0.5-2% is commonly used however we are suggesting a lower range up to 0.05% as the typical dermatological anesthetic component used is approximately 10% lower than what is used in the pharmaceutical applications. In addition a 5% solution is deemed safe as well from animal studies (Wilson & Wilson. 1999. Benzyl Alcohol as an Alternative Local Anesthetic. Annals of Emergency Medicine. 495-499) (European Medicines Agency, 9 Oct. 2017 EMA/CHMP/272866/2013 Committee for Human Medicinal Products (CHMP, Benzyl alcohol and benzoic acid group used as excipients).

Accordingly, the invention should be understood to be limited only by the accompanying claims, purposively construed.

Having described my invention, other and additional preferred embodiments will become apparent to those skilled in the art to which it pertains, and without deviating from the scope of the appended claims. The detailed description and drawings are further understood to be supportive of the disclosure, the scope of which being defined by the claims. While some of the best modes and other embodiments for carrying out the claimed teachings have been described in detail, various alternative designs and embodiments exist for practicing the disclosure defined in the appended claims.

The foregoing disclosure is further understood as not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, a person of ordinary skill in the art will recognize that changes may be made in form and detail without departing from the scope of the present disclosure. Thus, the present disclosure is limited only by the claims.

In the foregoing specification, the disclosure has been described with reference to specific embodiments. However, as one skilled in the art will appreciate, various embodiments disclosed herein can be modified or otherwise implemented in various other ways without departing from the spirit and scope of the disclosure. Accordingly, this description is to be considered as illustrative and is for the purpose of teaching those skilled in the art the manner of making and using various embodiments of the disclosure. It is to be understood that the forms of disclosure herein shown and described are to be taken as representative embodiments. Equivalent elements, materials, processes or steps may be substituted for those representatively illustrated and described herein. Moreover, certain features of the disclosure may be utilized independently of the use of other features, all as would be apparent to one skilled in the art after having the benefit of this description of the disclosure. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural.

Further, various embodiments disclosed herein are to be taken in the illustrative and explanatory sense, and should in no way be construed as limiting of the present disclosure. All joinder references (e.g., attached, affixed, coupled, connected, and the like) are only used to aid the reader's understanding of the present disclosure, and may not create limitations, particularly as to the position, orientation, or use of the systems and/or methods disclosed herein. Therefore, joinder references, if any, are to be construed broadly. Moreover, such joinder references do not necessarily infer that two elements are directly connected to each other.

Additionally, all numerical terms, such as, but not limited to, “first”, “second”, “third”, “primary”, “secondary”, “main” or any other ordinary and/or numerical terms, should also be taken only as identifiers, to assist the reader's understanding of the various elements, embodiments, variations and/or modifications of the present disclosure, and may not create any limitations, particularly as to the order, or preference, of any element, embodiment, variation and/or modification relative to, or over, another element, embodiment, variation and/or modification.

It will also be appreciated that one or more of the elements depicted in the drawings/figures can also be implemented in a more separated or integrated manner, or even removed or rendered as inoperable in certain cases, as is useful in accordance with a particular application. Additionally, any signal hatches in the drawings/figures should be considered only as exemplary, and not limiting, unless otherwise specifically specified.

1. A soft tissue filler composition comprising:
a cross-linked hyaluronic acid; and
benzyl alcohol.

2. The soft tissue filler composition of claim 1 wherein the benzyl alcohol is present at a concentration between about 0.1% and about 2.0% by weight of said composition.
